(19) United States
(12) Patent Application Publication
(10) Pub. No.: US 2025/0232437 A1
(43) Pub. Date: Jul. 17, 2025

(54) IMAGE DETERMINATION APPARATUS AND METHOD, AND COMPUTER-READABLE RECORDING MEDIUM STORING PROGRAM

(71) Applicant: Konica Minolta, Inc., Tokyo (JP)

(72) Inventors: Osamu Toyama, Hyogo (JP); Tatsuya Takagi, Tokyo (JP)

(73) Assignee: Konica Minolta, Inc., Tokyo (JP)

(21) Appl. No.: 19/020,310

(22) Filed: Jan. 14, 2025

(30) Foreign Application Priority Data
Jan. 17, 2024 (JP) 2024-005316

(51) Int. Cl. G06T 7/00; A61B 5/00; A61B 5/091; G06V 10/44; G16H 30/20; G16H 50/20

(52) U.S. Cl. CPC G06T 7/0012; A61B 5/091; A61B 5/7235; G06V 10/44; G16H 30/20; G16H 50/20; G06T 2207/30061

(57) ABSTRACT

An image determination apparatus according to an embodiment of the present disclosure includes: an image acquisition means that acquires a dynamic image obtained by capturing a site including a diagnosis target region of a patient; a feature amount extraction means that extracts a feature amount through a first process based on the dynamic image; a determination means that makes a determination related to diagnosis through a second process based on a result of machine learning based on the feature amount; and an explanation data generation means that generates explanation data based on the feature amount and the determination related to the diagnosis; and an outputter or a communicator that outputs the explanation data to outside.

### CROSS REFERENCE TO RELATED APPLICATIONS

The entire disclosure of Japanese Patent Application No. 2024-005316 filed on Jan. 17, 2024 is incorporated herein by reference in its entirety.

### BACKGROUND

### Technological Field

The present disclosure relates to an image determination apparatus and method, and a computer-readable recording media and storing programs.

### Description of Related Art

Class determination has been performed on medical images by machine learning to determine whether the images have been captured with appropriate positioning (e.g., Japanese Unexamined Patent Publication No. 2021-97864).

For the informed consent, a doctor, a nurse, or the like needs to explain a situation of a disease (a medical condition or a pathological condition), a treatment policy, and the like to a patient. The device of PTL 1 performs class determination on the basis of a feature amount, but does not output the feature amount that is the basis of the class determination. Therefore, even if the device of Patent Document 1 is used for determination of a disease, no explanatory material for informed consent is output. A medical image is an image captured using radiation or ultrasonic waves and used by a doctor or the like, but is unfamiliar to patients. Therefore, when a doctor or the like explains a grasped situation of a disease or a determined treatment policy to a patient, even if the doctor or the like explains using a medical image serving as a basis for determining the situation of the disease or the treatment policy, the explanation is not easy for the patient to understand in many cases.

Recently, a doctor determines diseases based on a dynamic image composed of a plurality of still images by repeatedly emitting radioactive pulses from a radioactive ray generating apparatus for a predetermined time (continuation time) in a cycle of a plurality of times per unit time (for example, 15 times per second) while an emission instruction is issued, and reading out the amount of charges generated in accordance with the amount of radioactive rays received through a subject as a signal value (intensity). That is, the dynamic image is a series of still images obtained by capturing a temporal change of the subject. The dynamic image may be an image of a 2D or an image of a 3D as long as a temporal change is photographed. In a case where the medical image is a dynamic image, since the dynamic image itself is novel, it may be difficult for a doctor to grasp an image feature caused by a pathological condition. It is more difficult for the doctor to give an easily understandable explanation because the patient is further unfamiliar with the medical image.

### SUMMARY

Therefore, in estimating the disease level of a specific disease by machine learning, it is required to assist the determination of a doctor and enable the doctor or the like to give an easy-to-understand explanation to a patient by providing the doctor with easy-to-understand data of a pathological condition or data necessary for explaining a treatment policy to the patient.

An image determination apparatus according to an embodiment of the present disclosure includes: a hardware processor that acquires a dynamic image obtained by capturing a site including a diagnosis target region of a patient; a hardware processor that extracts a feature amount through a first process based on the dynamic image; a hardware processor that makes a determination related to diagnosis through a second process based on a result of machine learning based on the feature amount; and a hardware processor that generates explanation data based on the feature amount and the determination related to the diagnosis; and an outputter or a communicator that outputs the explanation data to outside.

An image determination method according to an embodiment of the present disclosure includes: acquiring a dynamic image obtained by capturing a site including a diagnosis target region of a patient; extracting a feature amount through a first process based on the dynamic image; making a determination related to diagnosis through a second process based on a result of machine learning based on the feature amount; and generating explanation data based on the feature amount and the determination related to the diagnosis; and outputting the explanation data to outside.

A computer-readable recording medium according to an embodiment of the present disclosure stores an image determination program that causes a computer to execute: acquiring a dynamic image obtained by capturing a site including a diagnosis target region of a patient; extracting a feature amount through a first process based on the dynamic image; making a determination related to diagnosis through a second process based on a result of machine learning based on the feature amount; and generating explanation data based on the feature amount and the determination related to the diagnosis; and outputting the explanation data to outside.

Note that these generic or specific aspects may be implemented as a system, an apparatus, a method, an integrated circuit, a computer program, or a recording medium, or any combination of the system, the apparatus, the method, the integrated circuit, the computer program, and the recording medium.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating a configuration of an image determination apparatus;

FIG. 2 is a functional block diagram of a determination circuit;

FIG. 3 is a diagram illustrating a flowchart of a processing circuit diagnosing COPD;

FIG. 4 is a diagram illustrating another flowchart of the processing circuit when the diagnosis of COPD is performed;

FIG. 5 is a diagram illustrating a flowchart of a processing circuit when a Fallot diagnosis is performed;

FIG. 6 is a diagram illustrating a flowchart of a processing circuit when the diagnosis of the CTEPH is performed;

FIG. 7 is a diagram illustrating an output example of an outputter;

FIG. 8 is a diagram illustrating another output example of the outputter; and

FIG. 9 is a view illustrating still another example of outputting by the outputter.

### DETAILED DESCRIPTION OF EMBODIMENTS

Hereinafter, one or more embodiment s of the present invention will be described with reference to the drawings. However, the scope of the invention is not limited to the disclosed embodiments.

In the following, embodiments of the present disclosure will be described in detail with reference to the drawings as appropriate.

### 1. Image Determination Apparatus

First, a schematic configuration of an image determination apparatus 100 according to an embodiment of the present disclosure will be described.

### Configuration

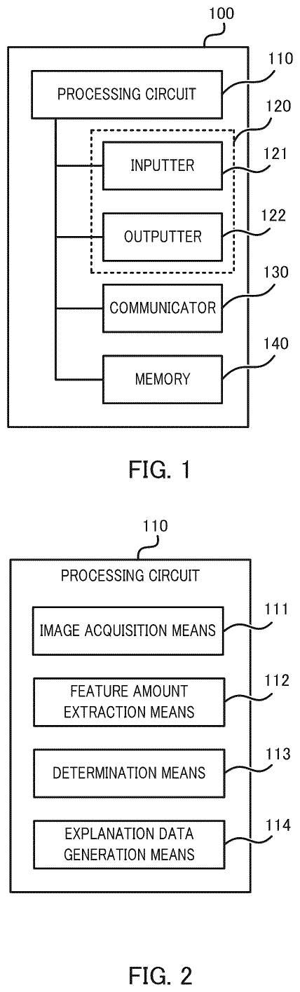

FIG. 1 is a diagram illustrating the configuration of the image determination apparatus 100.

The image determination apparatus 100 includes a processing circuit 110, an input/output unit 120, a communicator 130, and a memory 140. The input/output unit 120 includes an inputter 121 and an outputter 122. The inputter 121 and the outputter 122 may be integrally formed.

The processing circuit 110 is composed of a CPU (Central Processing Unit), a GPU (Graphics Processing Unit), an ASIC (Application Specific Integrated Circuit), an FPGA (Field Programmable Gate Array) and the like, and includes a neural network. The processing circuit 110 extracts a feature amount and estimates a disease level of a specific disease based on the input medical image. Details of the processing circuit 110 and will be described later.

The inputter 121 includes at least one of a touch panel, a keyboard, a mouse, a microphone, and the like, and inputs information based on an operation performed by a user (a doctor, a radiology technician, or the like).

The outputter 122 includes at least one of a display, a speaker, a printer, and the like, and outputs the explanation data generated by the processing circuit 110 to the outside.

The communicator 130 communicates with an external device by a wireless or wired bus, a Local Area Network (LAN), the Internet, a Virtual Private Network (VPN), a public line, or the like. The communicator 130 communicates with the hospital information systems (Hospital Information System: HIS), a radiology department information system (Radiology Information System: RIS), an image storage and communication system (Picture Archiving and Communication System: PACS) Communicate with a dynamic analysis device or the like.

The memory 140 is made up of a ROM (Read Only Memory), a RAM (Random Access Memory), an EPROM (Erasable Programmable ROM), an EEPROM (Electrically Erasable Programmable Read-Only Memory), an HDD (Hard Disk Drive), or the like, and stores medical images, learning datasets (training datasets), various programs, and the like.

FIG. 2 is a functional block diagram of the processing circuit 110.

The processing circuit 110 includes an image acquisition means 111, a feature amount extraction means 112, a determination means 113, and an explanation data generation means 114. The processing circuit 110 estimates the disease level of the disease by the two means of the feature amount extraction means 112 and the determination means 113.

The image acquisition means 111 acquires a medical image. The medical image is, for example, a dynamic image.

The feature amount extraction means 112 extracts, based on the medical image acquired by the image acquisition means 111, a feature amount (image) effective for a doctor or the like to give an explanation to the patient. The feature amount extraction means 112 extracts a feature amount. The feature amount may be numerical data or image data. The feature amount may be one still image making up the dynamic image. Since the feature amount extracted by the feature amount extraction means 112 is information necessary for the determination means 113 to estimate the disease level, it is important that the feature amount is information necessary for the determination means 113 to perform estimation with high accuracy, and the feature amount does not necessarily needs to be information that is easy for humans to understand. The details of the feature amount extraction means 112 will be described later.

The determination means 113 estimates the disease level of a specific disease based on the feature amount extracted by the feature amount extraction means 112. The determination means 113 outputs the estimated disease level. The details of the determination means 113 will be described later.

The explanation data generation means 114 generates, based on the feature amount extracted by the feature amount extraction means 112 and the disease level estimated by the determination means 113, explanation data that is information with which a doctor can easily grasp the situation of a disease or information useful for a doctor or the like to make an explanation for a patient. In the present disclosure, the explanation data is information with which a doctor or the like can easily grasp the situation of a disease or information useful for a doctor or the like to make an explanation for a patient. Even when the feature amount extracted by the feature amount extraction means 112 is not easy-to-understand information for humans, the description data generation means 114 can change it to easy-to-understand information for humans. The doctor or the like can grasp the situation of the disease by using the generated explanation data and explain the situation of the disease and the treatment policy to the patient. The explanation data generated by the explanation data generation means 114 is output from the outputter 122.

### Diagnosis of COPD: First Embodiment

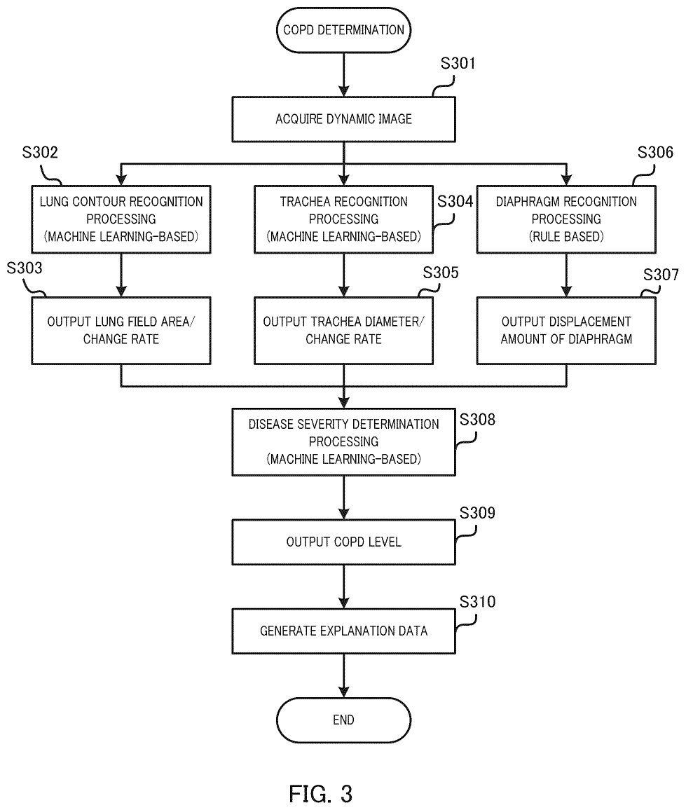

FIG. 3 is a diagram illustrating a flowchart of the processing circuit 110 diagnosing COPD (Chronic Obstructive Pulmonary Disease).

COPD affects the bronchi and alveoli. When determining the disease level of COPD, a doctor refers to the lung field area, the change rate of the lung field area, the trachea diameter, the change rate of the trachea diameter, the displacement amount of the diaphragm, the change amount of the alveoli, the image density, the variance of each change amount, and the like. The disease level of COPD may be stage (stage I, stage II, stage III, stage IV). The feature amount may be, for example, a value indicating at least one of the lung field area, the change rate of the lung field area, the trachea diameter, the change rate of the trachea diameter, the displacement amount of the diaphragm, the change amount of the alveoli, the image density, and the variance of each change amount, or one or more still images (a part of the dynamic image) making up the dynamic image for understanding these values or one obtained by processing one or more still images.

### Image Acquisition Means

The image acquisition means 111 acquires a medical image (step S301). The medical image is, for example, a dynamic image. The image acquisition means 111 may read the dynamic image stored in the memory 140 or may receive the dynamic image from the RIS or the like via the communicator 130.

### Feature Amount Extraction Means

The feature amount extraction means 112 extracts a feature amount from the dynamic images acquired by the image acquisition means 111 in step S301, based on machine learning or a rule, and outputs the extracted feature amount to the outside from the outputter 122, or outputs (transmits) the extracted feature amount to an external device via the communicator 130.

The feature amount extraction means 112 performs lung contour recognition processing based on machine learning on the dynamic image to obtain the lung field area and the change rate of the lung field area (step S302). As the preprocessing, edge processing may be performed on the dynamic image. The feature amount extraction means 112 may obtain the lung field area of each of the still images making up the dynamic image based on machine learning and obtain the change rate of the lung field area by arithmetic processing. An average, a maximum, a minimum, a median, or a mode of the lung field area of the respective still images may be set as the lung field area.

The feature amount extraction means 112 outputs the lung field area and the change rate of the lung field area extracted in step S302 to the outside (step S303). The feature amount extraction means 112 may output the maximum lung field area and/or the minimum lung field area.

The feature amount extraction means 112 performs a trachea recognition process based on machine learning on the dynamic image, and obtains a trachea diameter and a change rate of the trachea diameter (step S304). As the preprocessing, edge processing may be performed on the dynamic image. The feature amount extraction means 112 may obtain the trachea diameter of each still image making up the dynamic image based on machine learning and obtain the change rate of the trachea diameter by arithmetic processing. The average, maximum, minimum, median, or mode of the trachea diameters of the still images may be defined as the trachea diameter.

The feature amount extraction means 112 outputs the trachea diameter and the change rate of the trachea diameter extracted in step S304 to the outside (step S305). The feature amount extraction means 112 may output the maximum trachea diameter and/or the minimum trachea diameter to the outside.

The feature amount extraction means 112 performs diaphragmatic recognition processing based on a rule on the dynamic image (step S306). The feature amount extraction means 112 obtains the position of the diaphragm based on the rule with respect to each still image making up the dynamic image, and obtains the displacement amount of the diaphragm by arithmetic processing. For the recognition of the diaphragm, for example, line fitting is used. The feature amount extraction means 112 may obtain the position of the diaphragm on the basis of machine learning. In this case, the feature amount extraction means 112 may perform edge processing on the dynamic image as preprocessing.

The feature amount extraction means 112 outputs the displacement amount of the diaphragm extracted in step S306 to the outside (step S307).

The feature amount extraction means 112 executes at least one of steps S302, S304, and S306. The feature amount extraction means 112 may determine processing to be executed, in accordance with the feature amount to be extracted. For example, when the feature amount extraction means 112 extracts the lung field area, the change rate of the lung field area, the trachea diameter, the change rate of the trachea diameter, and the displacement amount of the diaphragm as the feature amount, three processes of a lung contour recognition process, a trachea recognition process, and a diaphragm recognition process are performed.

Machine learning performed by the feature amount extraction means 112 will be described.

In the learning phase, the feature amount extraction means 112 performs learning with learning data in which a feature amount for a dynamic image is used as teacher data. The feature amount extraction means 112 performs learning with learning data corresponding to each processing, for example, learning data for “lung contour recognition processing” and learning data for “trachea recognition process”. The learning may be learning corresponding to a doctor or the like. The learning may be learning corresponding to an attribute of a patient, for example, whether the patient is an adult or a child.

In the inference phase, the feature amount extraction means 112 extracts a feature amount from the dynamic image on the basis of the result learned in the learning phase.

### Determination Means

The determination means 113 estimates the COPD disease level based on the feature amounts extracted by the feature amount extraction means 112 in steps S302, S304, and S306 based on machine learning (step S308). If the feature amount extracted by the feature amount extraction means 112 is an impossible numerical value (e.g., when trachea diameter is 10 cm or the like), the determination means 113 may exclude the feature amount before performing estimation.

The determination means 113 outputs the COPD disease level estimated in step S308 to the outside (step S309). The determination means 113 may output the estimated disease level of COPD to the outside from the outputter 122, or may output (transmit) the estimated disease level to an external device via the communicator 130.

The machine learning performed by the determination means 113 will be described.

In the learning phase, the determination means 113 learns from learning data in which the disease level of COPD with respect to the feature amount is used as teacher data.

In the inference phase, the determination means 113 estimates the disease level of COPD from the feature amount on the basis of the result learned in the learning phase.

### Explanation Data Generation Unit

The explanation data generation means 114 generates, based on the feature amount extracted by the feature amount extraction means 112 and the disease level estimated by the determination means, explanation data that allows a doctor or the like to easily grasp the status of the disease and data that is used by a doctor or the like to explain the status of the disease and a treatment policy for the patient (step S310).

The explanation data to be generated may be a still image making up the dynamic image acquired by the image acquisition means 111 or an image obtained by subjecting the dynamic image to image processing, or may be data to which an annotation, a marking, a numerical value of a feature amount, or the like has been added. For example, a line indicating the lung field region may be added to the still image, or a numerical value such as the lung field area may be added, as the explanation data.

### Diagnosis of COPD: Second Embodiment

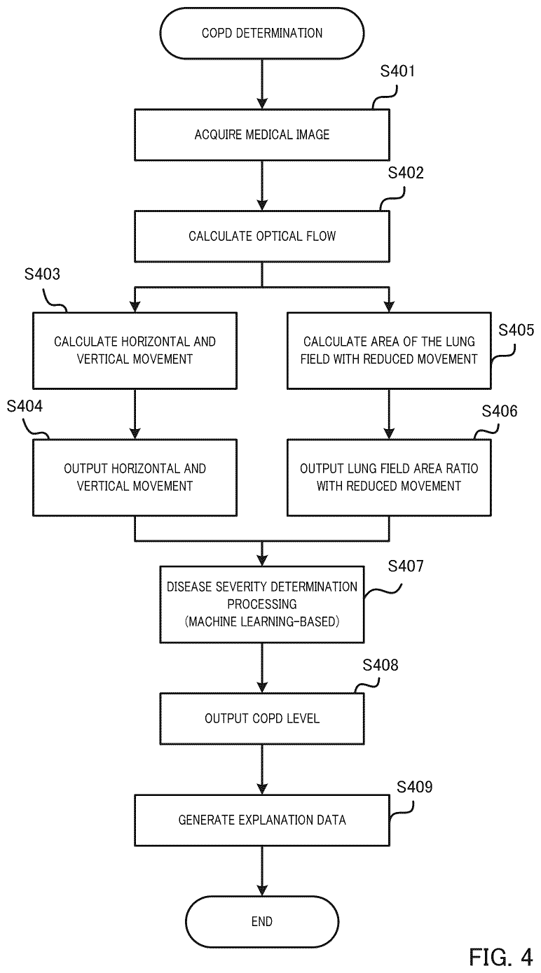

FIG. 4 is a diagram illustrating another flowchart of the processing circuit 110 diagnosing COPD. The disease level of COPD may be stage (stage I, stage II, stage III, stage IV). The feature amount may be, for example, a value indicating at least one of the ratio of the magnitude of the movement of each point of the lung field, the area of the entire lung field, the area of the lung field with reduced movement, and the ratio of the area of the lung field with reduced movement to the area of the entire lung field, or one or more still images (a part of the dynamic image) making up the dynamic image for understanding these values or one obtained by processing one or more still images.

### Image Acquisition Means

The image acquisition means 111 acquires a medical image (step S401). The medical image is, for example, a dynamic image. The image acquisition means 111 may read the dynamic image stored in the memory 140 or may receive the dynamic image from the RIS or the like via the communicator 130.

### Feature Amount Extraction Means

The feature amount extraction means 112 extracts a feature amount from the dynamic images acquired by the image acquisition means 111 in step S401, based on machine learning, and outputs the extracted feature amount to the outside from the outputter 122, or outputs (transmits) the extracted feature amount to an external device via the communicator 130.

The feature amount extraction means 112 calculates an optical flow (difference) from the dynamic images acquired by the image acquisition means 111 in step S401 (step S402). The difference may be a difference with respect to a reference frame (still image) or a difference with respect to an immediately preceding frame. The reference frame may be an initial frame. The difference is calculated as a vector. The step S402 is performed as preprocessing.

The feature amount extraction means 112 specifies the lung fields based on the optical flow calculated in step S402 and based on machine learning. The feature amount extraction means 112 calculates the size of the entire lung field in the horizontal/vertical directions. In addition, since the optical flow is a vector, the magnitude of the movement can be grasped by the magnitude of the maximum vector in a predetermined period in a case where the optical flow is a difference from the reference frame, and by the sum of the magnitudes of the vectors in the predetermined period in a case where the optical flow is a difference from the immediately preceding frame. The feature amount extraction means 112 calculates, for each point in the lung field, the magnitude of the movement in the horizontal/vertical direction (i.e., the magnitude of the vector in the horizontal/vertical direction) based on the rule. Based on the size of the entire lung fields in the horizontal/vertical direction and the magnitude of the movement of each point in the horizontal/vertical direction, the feature amount extraction means 112 calculates the ratio of the magnitude of the movement of each point in the horizontal/vertical direction to the size of the entire lung fields in the horizontal/vertical direction (step S403).

The feature amount extraction means 112 outputs the ratio of the magnitude of the movement for each point of the lung fields calculated in step S403 to the outside (step S404). The feature amount extraction means 112 may output the ratio of the magnitudes of the movements of some of the spots.

Based on the optical flow calculated in step S402, the feature amount extraction means 112 calculates the ratio of the area of the lung field with reduced movement based on machine learning (step S405). The feature amount extraction means 112 may calculate the ratio of the area of the lung field with reduced movement, based on the ratio of the magnitudes of the movements of the lung fields at the respective points calculated in step S403. For example, when the ratio of the magnitude of the movement is equal to or smaller than a predetermined threshold value, it may be determined that the movement is reduced, and the ratio of the area of the lung field with reduced movement may be calculated based on the number of points with reduced movement and the number of points in the entire lung field.

The feature amount extraction means 112 outputs, to the outside, the areas of the entire lung fields, the area of the lung field with reduced movement, and the ratios of the area of the lung field with reduced movement to the areas of the entire lung fields, which are calculated in step S405 (step S406).

Machine learning performed by the feature amount extraction means 112 will be described.

In the learning phase, the feature amount extraction means 112 performs learning with learning data in which a feature amount for an optical flow is used as teacher data. The feature amount extraction means 112 performs learning using learning data corresponding to each processing, for example, learning data for “lung contour recognition processing” and learning data for “calculation of area of the lung field with reduced movement”. The learning may be learning corresponding to a doctor or the like. The learning may be learning corresponding to an attribute of a patient, for example, whether the patient is an adult or a child.

In the inference phase, the feature amount extraction means 112 extracts a feature amount from the optical flow on the basis of the result learned in the learning phase.

### Determination Means

The determination means 113 estimates the COPD level based on the feature amount extracted by the feature amount extraction means 112 in steps S403 and S405 based on machine learning (step S407). If the feature amount extracted by the feature amount extraction means 112 is an impossible numerical value (e.g., if the size of the entire lung field is 5 cm² or the like), the determination means 113 may exclude the feature amount before the estimation.

The determination means 113 outputs the COPD disease level estimated in step S407 to the outside (step S408). The determination means 113 may output the estimated disease level of COPD to the outside from the outputter 122, or may output (transmit) the estimated disease level to an external device via the communicator 130.

The machine learning performed by the determination means 113 will be described.

In the learning phase, the determination means 113 learns from learning data in which the disease level of COPD with respect to the feature amount is used as teacher data.

In the inference phase, the determination means 113 estimates the disease level of COPD from the feature amount on the basis of the result learned in the learning phase.

### Explanation Data Generation Unit

The explanation data generation means 114 generates, based on the feature amount extracted by the feature amount extraction means 112 and the disease level estimated by the determination means, explanation data that allows a doctor or the like to easily grasp the status of the disease and data that is used by a doctor or the like to explain the status of the disease and a treatment policy for the patient (step S409).

The explanation data to be generated may be a still image making up the dynamic image acquired by the image acquisition means 111 or an image obtained by subjecting the dynamic image to image processing, or may be data to which an annotation, a marking, a numerical value of a feature amount, or the like has been added. For example, the explanation data may be obtained by adding, to the still image, a line indicating the region of the lung field with reduced movement or adding numerical values such as a ratio and an area.

### Diagnosis of Fallot: Third Embodiment

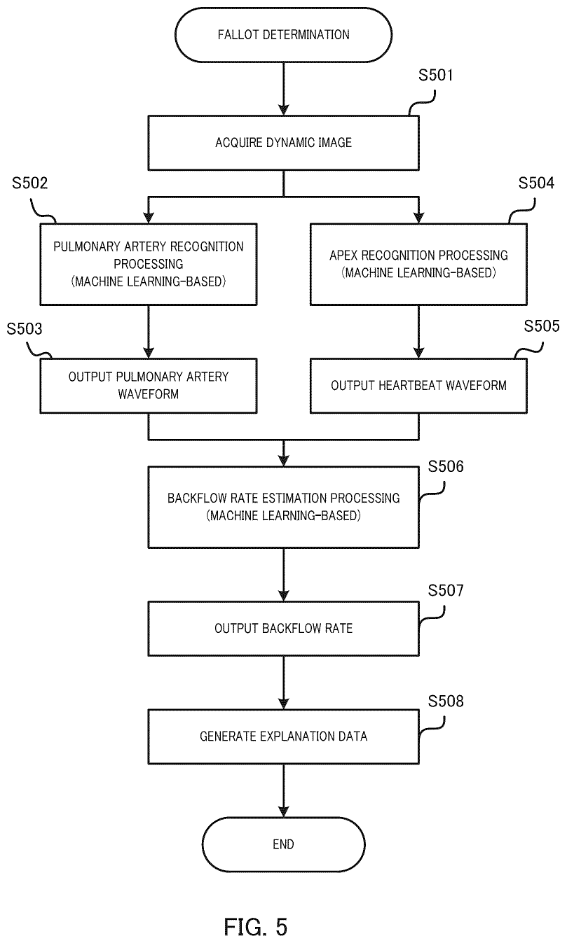

FIG. 5 is a diagram illustrating a flowchart of the processing circuit 110 diagnosing Fallot (tetralogy of Fallot).

Fallot (tetralogy of Fallot) affects the heart. A doctor or the like uses a feature amount such as a pulmonary artery waveform or a heartbeat waveform in the Fallot determination. The disease level of Fallot may be backflow rate or NYHA (New York Heart Association) cardiac function classifications (stage I, stage II, stage III, and stage IV). The feature amount may be, for example, a value indicating at least one of the waveform of the pulmonary artery and the heartbeat waveform, or one or more still images (a part of the dynamic image) making up the dynamic image for understanding these values, or one obtained by processing one or more still images. For example, a line indicating the pulmonary artery may be added to the still image to obtain the feature amount.

### Image Acquisition Means

The image acquisition means 111 acquires a medical image (step S501). The medical image is, for example, a dynamic image. The image acquisition means 111 may read the dynamic image stored in the memory 140 or may receive the dynamic image from the RIS or the like via the communicator 130.

### Feature Amount Extraction Means

The feature amount extraction means 112 extracts a feature amount from the dynamic images acquired by the image acquisition means 111 in step S501, based on machine learning, and outputs the extracted feature amount to the outside from the outputter 122, or outputs (transmits) the extracted feature amount to an external device via the communicator 130.

The feature amount extraction means 112 performs recognition processing of the pulmonary arteries based on machine learning on the dynamic image, and extracts the waveform of the pulmonary arteries (step S502). The feature amount extraction means 112 may extract waveforms of the pulmonary artery at a plurality of points, for example, a point close to the heart and a point close to the lungs in the recognized pulmonary artery. The feature amount extraction means 112 recognizes the pulmonary artery in each still image making up the dynamic image based on machine learning, selects a plurality of points of the recognized pulmonary artery, and detects the waveform of the pulmonary artery at each point as a change in density corresponding to time. The feature amount extraction means 112 may recognize a plurality of points of the pulmonary artery in each still image making up the dynamic image on the basis of machine learning.

The feature amount extraction means 112 outputs the waveform of the pulmonary arteries extracted in step S502 to the outside (step S503). The feature amount extraction means 112 may output the waveform of the pulmonary artery at some points to the outside.

The feature amount extraction means 112 performs a process of recognizing the apex based on machine learning on the dynamic image and extracts the waveform of the apex, that is, the heartbeat waveform (step S504). The feature amount extraction means 112 obtains the apex in each of the still images forming the dynamic image based on machine learning, and detects the heartbeat waveform as a change in density corresponding to time.

The feature amount extraction means 112 outputs the heartbeat waveform extracted in step S504 to the outside (step S505).

The feature amount extraction means 112 may perform only one of steps S502 and S504. When extracting the waveforms at a plurality of points of the pulmonary artery, the recognition processing of the pulmonary artery may be performed for each point.

Machine learning performed by the feature amount extraction means 112 will be described.

In the learning phase, the feature amount extraction means 112 performs learning with learning data in which a feature amount for a dynamic image is used as teacher data. The feature amount extraction means 112 performs learning using learning data corresponding to each processing, for example, learning data for “pulmonary artery recognition processing” and learning data for “cardiac apex recognition processing”. The feature amount may be numerical data or image data. The learning may be learning corresponding to a doctor or the like. The learning may be learning corresponding to an attribute of a patient, for example, whether the patient is an adult or a child.

In the inference phase, the feature amount extraction means 112 extracts feature amounts from the dynamic images on the basis of machine learning.

### Determination Means

The determination means 113 estimates the disease level of Fallot based on the feature amounts extracted by the feature amount extraction means 112 in steps S502 and S504 based on machine learning (step S506). If the feature amount extracted by the feature amount extraction means 112 is an impossible numerical value (e.g., if the number of heartbeats is 500 times per minute), the determination means 113 may exclude the feature amount before estimation is performed.

The determination means 113 outputs the disease level of Fallot estimated in step S506 to the outside (step S507). The determination means 113 may output the estimated disease level of Fallot to the outside from the outputter 122, or may output (transmit) the disease level to an external device via the communicator 130.

In the learning phase, the determination means 113 learns from learning data in which the disease level of Fallot with respect to a feature amount is used as teacher data.

In the inference phase, the determination means 113 estimates the disease level of Fallot from the feature amount on the basis of the result learned in the learning phase.

### Explanation Data Generation Unit

The explanation data generation means 114 generates, based on the feature amount extracted by the feature amount extraction means 112 and the disease level estimated by the determination means, explanation data that allows a doctor or the like to easily grasp the status of the disease and data that is used by a doctor or the like to explain the status of the disease and a treatment policy for the patient (step S508).

The explanation data to be generated may be a still image making up the dynamic image acquired by the image acquisition means 111 or an image obtained by subjecting the dynamic image to image processing, or may be data to which an annotation, a marking, a numerical value of a feature amount, or the like has been added. For example, a line indicating the pulmonary artery may be added to the still image to be the explanation data.

### Diagnosis of CTEPH: Fourth Embodiment

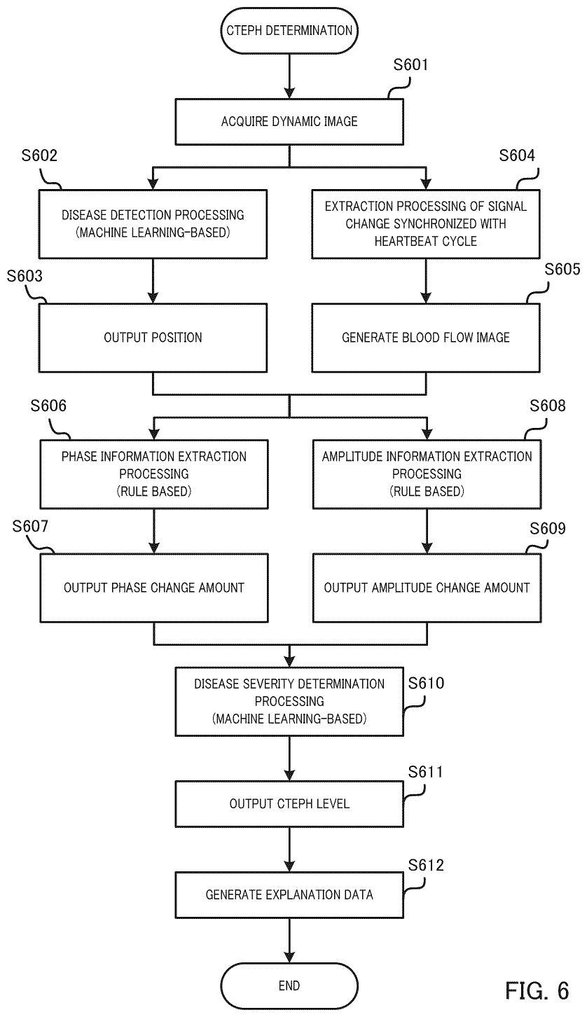

FIG. 6 is a diagram illustrating a flowchart of the processing circuit 110 diagnosing CTEPH (Chronic Thrombo Embolic Pulmonary Hypertension).

CTEPH affects the lungs and the heart. The doctor or the like uses the blood flow image, the phase change amount and the amplitude change amount for each measurement position in the determination of the CTEPH. The disease level of CTEPH may be a certainty factor or may be NYHA functional categories (stage I, stage II, stage III, and stage IV) and/or World Health Organization (WHO) pulmonary hypertension functional categories (degree I, degree II, degree III, and degree IV). The feature amount may be, for example, a value indicating at least one of the phase change amount and the amplitude change amount of the blood flow image at each measurement position, one or more still images (a part of the dynamic image) making up the dynamic image for understanding these values, or one obtained by processing one or more still images.

### Image Acquisition Means

The image acquisition means 111 acquires a medical image (step S601). The medical image is, for example, a dynamic image. The image acquisition means 111 may read the dynamic image stored in the memory 140 or may receive the dynamic image from the RIS or the like via the communicator 130.

### Feature Amount Extraction Means

The feature amount extraction means 112 extracts a feature amount from the dynamic images acquired by the image acquisition means 111 in step S601, based on machine learning, and outputs the extracted feature amount to the outside from the outputter 122, or outputs (transmits) the extracted feature amount to an external device via the communicator 130.

The feature amount extraction means 112 identifies the positions of the organ and the lung fields in the dynamic image on the basis of machine learning, and identifies the reference positions and the measurement positions for extracting the amplitude information and the phase information on the basis of the identified positions of the organ and the lung fields (step S602). For example, the reference position may be the hilum of the lung (a position with a high blood flow rate connected to the heart), and the measurement positions may be the right upper lobe, the right middle lobe, the right lower lobe, the left upper lobe, and the left lower lobe. The reference position may be a maximum point of the signal change in the entire.

The feature amount extraction means 112 outputs the position extracted in step S602 to the outside (step S603). The feature amount extraction means 112 may output the positions of some of the spots to the outside.

The feature amount extraction means 112 performs extraction processing of a signal change synchronized with a heartbeat cycle on the dynamic image on the basis of machine learning, and generates a blood flow image (step S604). The feature amount extraction means 112 obtains a heartbeat cycle from the dynamic image on the basis of machine learning, and extracts a change in a signal synchronized with the heartbeat cycle.

The feature amount extraction means 112 outputs the blood flow image generated in step S604 to the outside (step S605).

The feature amount extraction means 112 performs, for each of the positions identified in step S602, phase information extraction processing on the blood flow image (change in signal synchronized with the heartbeat cycle) generated in step S604 and extracts a phase change amount (step S606). The feature amount extraction means 112 extracts a phase change amount based on a rule. The feature amount extraction means 112 compares the phase data at the reference position with the phase data at each measurement position and calculates a phase change amount (i.e., a delay time) at each measurement position.

The feature amount extraction means 112 outputs the phase change amount generated in step S606 to the outside (step S607). The feature amount extraction means 112 may output the phase change amount at some points to the outside.

The feature amount extraction means 112 performs, for each of the positions identified in step S602, amplitude information extraction processing on the amplitudes of the blood flow images (changes in signals synchronized with the heartbeat cycle) generated in step S604 and extracts the amplitude change amount (step S608). The feature amount extraction means 112 extracts an amplitude change amount on the basis of a rule. The feature amount extraction means 112 compares the amplitude data at the reference position with the amplitude data at each measurement position and calculates an amplitude change amount (i.e., attenuation) at each measurement position.

The feature amount extraction means 112 outputs the amplitude change amount generated in step S608 to the outside (step S609). The feature amount extraction means 112 may output the amplitude change amount at some of the points to the outside.

Machine learning performed by the feature amount extraction means 112 will be described.

In the learning phase, the feature amount extraction means 112 performs learning with learning data in which a feature amount for a dynamic image is used as teacher data. The feature amount may be numerical data or image data. The learning may be learning corresponding to a doctor or the like. The learning may be learning corresponding to an attribute of a patient, for example, whether the patient is an adult or a child.

In the inference phase, the feature amount extraction means 112 extracts a feature amount from the dynamic image on the basis of the result learned in the learning phase.

### Determination Means

The determination means 113 estimates the disease level of CTEPH on the basis of machine learning, based on the feature amount extracted by the feature amount extraction means 112 (step S610). In a case where the feature amount extracted by the feature amount extraction means 112 is an impossible numerical value, the determination means 113 may exclude the feature amount before performing the estimation.

The determination means 113 outputs the disease level of CTEPH estimated in step S610 to the outside (step S611). The determination means 113 may output the estimated disease level of CTEPH to the outside from the outputter 122, or may output (transmit) it to an external device via the communication part 130.

The machine learning performed by the determination means 113 will be described.

In the learning phase, the determination means 113 performs learning using learning data in which the disease level of CTEPH for the feature amount is set as teacher data.

In the inference phase, the determination means 113 estimates the disease level of CTEPH from the feature amount on the basis of the result learned in the learning phase.

### Explanation Data Generation Unit

The explanation data generation means 114 generates, based on the feature amount extracted by the feature amount extraction means 112 and the disease level estimated by the determination means, explanation data that allows a doctor or the like to easily grasp the status of the disease and data that is used by a doctor or the like to explain the status of the disease and a treatment policy for the patient (step S612).

The explanation data to be generated may be a still image making up the dynamic image acquired by the image acquisition means 111 or an image obtained by subjecting the dynamic image to image processing, or may be data to which an annotation, a marking, a numerical value of a feature amount, or the like has been added. For example, data obtained by adding a mark indicating the measurement position to the still image may be used as the explanation data.

### Output

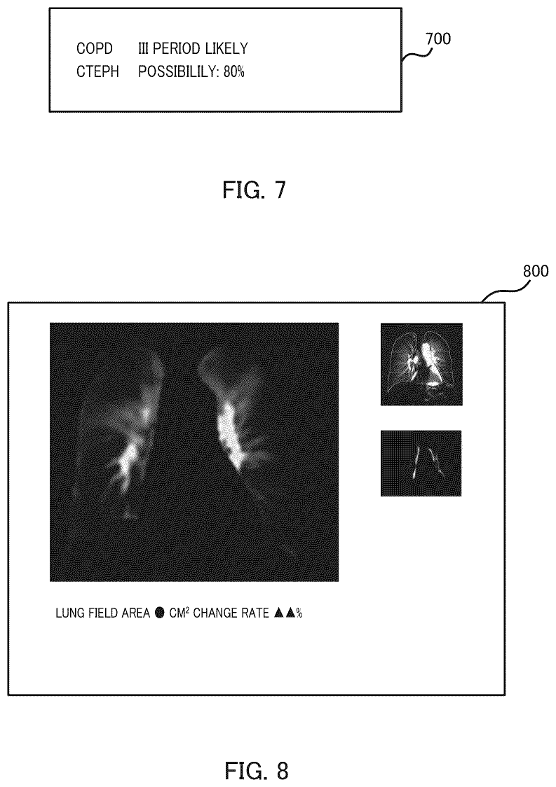

FIGS. 7 to 9 are diagrams illustrating output examples of the outputter 122. The image data may be transmitted to an external device via the communicator 130, and the image data in FIG. 7, 8, or 9 may be output by the external device. The outputter 122 may be any output as long as explanation data for a doctor to make an explanation for a patient is output.

The disease for which the image determination apparatus determines the disease level may be selected by a doctor or the like, may be determined in accordance with the patient (medical record), or may be all diseases that can be determined.

When the feature amount cannot be extracted due to the resolution or the like of the medical information, a message indicating that the determination cannot be performed may be output from the outputter 122 or the communicator 130 to the outside.

### Output Example 1

FIG. 7 is a diagram illustrating an output example of the outputter 122. A window 700 may be displayed on the display. FIG. 7 illustrates an example in which the disease levels of COPD and CTEPH are determined.

The disease level of the disease determined by the determination means 113 is output as the window 700. Disease levels of a plurality of diseases may be output.

### Output Example 2

FIG. 8 is a diagram illustrating another output example of the outputter 122. A window 800 may be displayed on the display.

The feature amount extraction means 112 outputs the extracted feature amount as the window 800. The explanation data generation means 114 may output an image obtained by performing image processing on the dynamic image and a plurality of feature amounts. A feature amount selected from among a plurality of feature amounts may be displayed in a large size. The feature amount may be displayed on a display for a patient based on an operation of a doctor or the like.

### Output Example 3

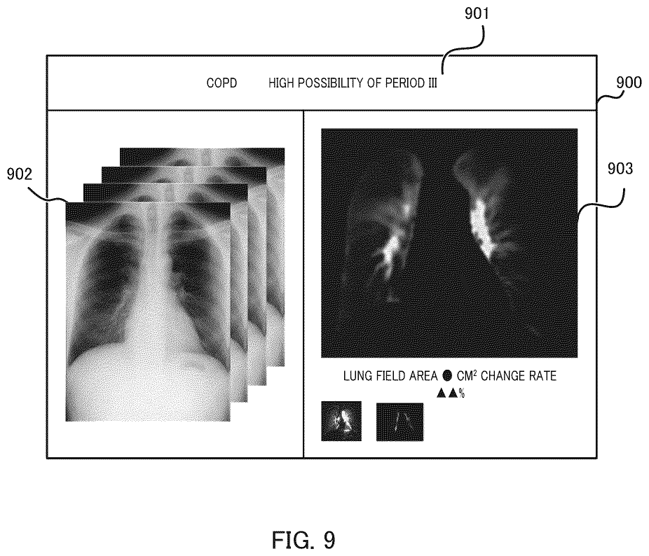

FIG. 9 is a diagram illustrating still another output example of the outputter 122. A window 900 may be displayed on the display.

The disease level 901 of the disease determined by the determination means 113, the medical image 902, the image 903 obtained by performing image processing on the dynamic image, and the feature amount (numerical data) may be displayed in the window 900. The disease level 901 of the disease, the medical image 902, the image 903 obtained by performing image processing on the dynamic image, and the feature amount may be displayed in one window 900 or may be displayed in different windows. The images or feature amounts obtained by performing image processing on the dynamic images may be displayed such that images or feature amounts obtained by performing a plurality of image processing on the dynamic images can be selected. FIG. 9 illustrates an example in which an image obtained by performing selected image processing on a dynamic image is displayed large and an image obtained by performing other image processing on the dynamic image is displayed small.

The outputter 122 may include a plurality of displays. For example, different information may be displayed on different displays, such as FIG. 9 being displayed on the display for the doctor and FIG. 8 being displayed on the display for the patient. The same information may be displayed on different displays.

When a doctor or the like explains a treatment policy or the like to a patient, it is possible to make an easy to understand explanation by using the output feature amount. The feature amount may be at least one of the examples, but it is possible to further improve the accuracy of the determination process by extracting and outputting many feature amounts.

Although the embodiments have been described above with reference to the drawings, the present disclosure is not limited to such examples. It is obvious that a person skilled in the art can conceive of various change examples or modification examples within the scope described in the claims. It is to be understood that such changes or modifications also belong to the technical scope of the present disclosure. Furthermore, the constituent elements in the embodiments may be combined as appropriate without departing from the spirit of the present disclosure.

(1) An image determination apparatus according to an embodiment of the present disclosure includes: a hardware processor that acquires a dynamic image obtained by capturing a site including a diagnosis target region of a patient; a hardware processor that extracts a feature amount through a first process based on the dynamic image; a hardware processor that makes a determination related to diagnosis through a second process based on a result of machine learning based on the feature amount; and a hardware processor that generates explanation data based on the feature amount and the determination related to the diagnosis; and an outputter or a communicator that outputs the explanation data to outside.

(2) In the image determination apparatus (1) according to the embodiment of the present disclosure, the first process is a process based on machine learning or a process based on a rule.

(3) In the image determination apparatus (1) according to the embodiment of the present disclosure, the dynamic image is a radiographic image acquired by a radiography apparatus.

(4) In the image determination apparatus (1) according to the embodiment of the present disclosure, the feature amount includes one or more still images making up the dynamic image.

(5) In the image determination apparatus (1) according to the embodiment of the present disclosure, the determination related to the diagnosis is determination of a disease level of a specific disease.

(6) In the image determination apparatus (5) according to the embodiment of the present disclosure, the specific disease is COPD; the disease level is a stage of the disease; and the feature amount is at least one of a lung field area, a change rate of the lung field area, a trachea diameter, a change rate of the trachea diameter, a displacement amount of a diaphragm, a change amount of alveoli, an image density, a variance of each change amount, one or more still images making up the dynamic image, and one or more processed still images.

(7) In the image determination apparatus (5) according to the embodiment of the present disclosure, the specific disease is COPD; the disease level is a stage of the disease; and the feature amount is at least one of a ratio of a magnitude of a movement for each point of a lung field, an area of the entire lung field, an area of the lung field with reduced movement, a ratio of the area of the lung field with reduced movement to the area of the entire lung field, one or more still images making up the dynamic image, and one or more processed still images.

(8) In the image determination apparatus (5) according to the embodiment of the present disclosure, the specific disease is tetralogy of Fallot; the disease level is a backflow rate; and the feature amount is at least one of a waveform of a pulmonary artery, a heartbeat waveform, one or more still images making up the dynamic image, and one or more processed still images.

(9) In the image determination apparatus (5) according to the embodiment of the present disclosure, the specific disease is CTEPH; the disease level is a certainty factor; and the feature amount is at least one of a phase change amount and an amplitude change amount of a blood flow image at each measurement position, one or more still images making up the dynamic image, and one or more processed still images.

(10) An image determination method according to an embodiment of the present disclosure includes: acquiring a dynamic image obtained by capturing a site including a diagnosis target region of a patient; extracting a feature amount through a first process based on the dynamic image; making a determination related to diagnosis through a second process based on a result of machine learning based on the feature amount; and generating explanation data based on the feature amount and the determination related to the diagnosis; and outputting the explanation data to outside.

(11) A computer-readable recording medium according to an embodiment of the present disclosure stores an image determination program that causes a computer to execute: acquiring a dynamic image obtained by capturing a site including a diagnosis target region of a patient; extracting a feature amount through a first process based on the dynamic image; making a determination related to diagnosis through a second process based on a result of machine learning based on the feature amount; and generating explanation data based on the feature amount and the determination related to the diagnosis; and outputting the explanation data to outside.

### INDUSTRIAL APPLICABILITY

The present disclosure is useful for an image determination apparatus.

Although embodiments of the present invention have been described and illustrated in detail, the disclosed embodiments are made for purpose of illustration and example only and not limitation. The scope of the present invention should be interpreted by terms of the appended claims.

What is claimed is:

1. An image determination apparatus comprising:
a hardware processor that acquires a dynamic image obtained by capturing a site including a diagnosis target region of a patient;
a hardware processor that extracts a feature amount through a first process based on the dynamic image;
a hardware processor that makes a determination related to diagnosis through a second process based on a result of machine learning based on the feature amount; and
a hardware processor that generates explanation data based on the feature amount and the determination related to the diagnosis; and
an outputter or a communicator that outputs the explanation data to outside.

2. The image determination apparatus according to claim 1, wherein the first process is a process based on machine learning or a process based on a rule.

3. The image determination apparatus according to claim 1, wherein the dynamic image is a radiographic image acquired by a radiography apparatus.

4. The image determination apparatus according to claim 1, wherein the feature amount includes one or more still images making up the dynamic image.

5. The image determination apparatus according to claim 1, wherein the determination related to the diagnosis is determination of a disease level of a specific disease.

6. The image determination apparatus according to claim 5,
wherein the specific disease is COPD;
wherein the disease level is a stage of the disease; and
wherein the feature amount is at least one of a lung field area, a change rate of the lung field area, a trachea diameter, a change rate of the trachea diameter, a displacement amount of a diaphragm, a change amount of alveoli, an image density, a variance of each change amount, one or more still images making up the dynamic image, and one or more processed still images.

7. The image determination apparatus according to claim 5,
wherein the specific disease is COPD;
wherein the disease level is a stage of the disease; and
wherein the feature amount is at least one of a ratio of a magnitude of a movement for each point of a lung field, an area of the entire lung field, an area of the lung field with reduced movement, a ratio of the area of the lung field with reduced movement to the area of the entire lung field, one or more still images making up the dynamic image, and one or more processed still images.

8. The image determination apparatus according to claim 5,
wherein the specific disease is tetralogy of Fallot;
wherein the disease level is a backflow rate; and
wherein the feature amount is at least one of a waveform of a pulmonary artery, a heartbeat waveform, one or more still images making up the dynamic image, and one or more processed still images.

9. The image determination apparatus according to claim 5,
wherein the specific disease is CTEPH;
wherein the disease level is a certainty factor; and
wherein the feature amount is at least one of a phase change amount and an amplitude change amount of a blood flow image at each measurement position, one or more still images making up the dynamic image, and one or more processed still images.

10. An image determination method comprising:
acquiring a dynamic image obtained by capturing a site including a diagnosis target region of a patient;
extracting a feature amount through a first process based on the dynamic image;
making a determination related to diagnosis through a second process based on a result of machine learning based on the feature amount; and
generating explanation data based on the feature amount and the determination related to the diagnosis; and
outputting the explanation data to outside.

11. A computer-readable recording medium storing an image determination program that causes a computer to execute:
acquiring a dynamic image obtained by capturing a site including a diagnosis target region of a patient;
extracting a feature amount through a first process based on the dynamic image;
making a determination related to diagnosis through a second process based on a result of machine learning based on the feature amount; and
generating explanation data based on the feature amount and the determination related to the diagnosis; and
outputting the explanation data to outside.
